(19) United States
(12) Patent Application Publication
(10) Pub. No.: US 2026/0250637 A1
(43) Pub. Date: Aug. 27, 2026

(54) PROCESS FOR PREPARATION OF HUMAN UMBILICAL CORD BLOOD PLASMA AND CULTURE MEDIA FOR TISSUE REGENERATION

(71) Applicant: ICETA - INSTITUTO DE CIÊNCIAS, TECNOLOGIAS E AGROAMBIENTE DA UNIVERSIDADE DO PORTO, Porto (PT)

(72) Inventor: Ana Colette MAURICIO, Porto (PT)

(21) Appl. No.: 18/853,144

(22) PCT Filed: Mar. 21, 2023
(86) PCT No.: PCT/IB2023/052753

§ 371 Date: Oct. 1, 2024

(30) Foreign Application Priority Data
Apr. 28, 2022 (PT) 117939

(51) Int. Cl. C12N 5/0775; A61K 35/51

(52) U.S. Cl. CPC C12N 5/0665; A61K 35/51; C12N 2501/11; C12N 2501/135; C12N 2501/15; C12N 2501/165; C12N 2501/22

(57) ABSTRACT

The present invention relates to a process for preparation of compositions comprising human umbilical cord blood plasma and a designed specific culture medium to grow these cells in specific conditions.

The compositions of the present invention are rich in regenerative factors derived from mesenchymal stem cells and together with other designed culture medium factors and processes, are useful in tissue regeneration processes and for preservation, conservation and expansion of mesenchymal stem cells. In fact, the present invention allows a better therapeutic option compared to traditional cell transplantation, as it can benefit from the local tissue response to the secreted molecules without the difficulties and complications associated to the engraftment of the allo- or xeno-transplanted mesenchymal stem cells.

Therefore, the present invention is in the field of cell-based therapies for skeletal disorders, such as joint disorders, disorders of connective tissue, bone diseases, osteoporosis, and other damaged tissues, and for modulating the inflammatory process related to tissue regeneration as well.

### TECHNICAL FIELD

The present invention relates to a process for preparation of compositions comprising human umbilical cord blood plasma (hUCBP) and a designed specific culture medium (CM) to grow these cells in specific conditions.

The present invention also relates to compositions comprising such regenerative factors and the specific culture media used for their preparation.

The compositions of the present invention are useful in tissue regeneration processes and for preservation and conservation of mesenchymal stem cells for clinical application.

Therefore, the present invention is in the field of cell-based therapies for skeletal disorders, such as joint disorders, disorders of connective tissue, bone diseases, osteoporosis, and other damaged tissues, and also for modulating the inflammatory process related to regeneration of tissues, in improved conditions.

### PRIOR ART

Mesenchymal Stromal/Stem Cells (MSCs) are at the forefront of research for the development of cell-based therapies, due to their capacity to self-renew and differentiate into several cell types, to secret soluble factors with paracrine actions, as well as due to their immunosuppressive and immunomodulatory properties.

A dominant part of these beneficial effects is assigned to the secretory capacity of these cells, through which they produce and release an array of bioactive molecules that impact the course of tissue healing.

The classical and widely accepted definition of MSCs is the one proposed by the International Society for Cellular Therapy (ISCT), wherein Mesenchymal Stem Cells are the ones, when ex-vivo cultured, which promote plastic adherence, expression of cluster of differentiation 105 (CD105), CD73 and CD90 surface molecules, absence of hematopoietic markers (CD45, CD14, CD19, CD34 and HLA-DR), and the capacity for at least, tri-lineage differentiation (into adipocytic, osteocytic and chondrocytic lineages).

Currently, it is possible to isolate human MSCs (hMSCs) from various sources like bone marrow, adipose tissue, skeletal muscle, umbilical cord blood (UCB), umbilical cord tissue or matrix (UCT, Wharton's jelly), peripheral blood, dental pulp, and amniotic fluid in classical and well-established cell culture conditions that includes MSCs isolation and in vitro expansion with growth factor supplementation with foetal bovine serum (FBS), and other animal sera.

Expansion of the hMSCs in vitro is essential to achieve appropriate cell numbers for clinical use and in vitro culture must be scale-up for clinical application purposes.

Increasing numbers of high-quality cryopreserved samples worldwide, in private and public cryopreservation banks, and the ready availability for therapeutic usage adds a further clinical advantage, as disclosed in Bieback et al., 2009, Huang et al., 2011, Hong et al., 2014), but addressing many of the disadvantages of the fetal bovine serum (FBS) used as culture medium supplement for scale-up purposes and for further clinical application.

Most of the times FBS and other animal sera have been used for culture media supplementation. However, the use of animal sera has several disadvantages including economic, ethical and scientific ones, and thus it is important to define alternatives for hMSCs in vitro culture with these products. These include chemically defined media, human serum, UCB serum r plasma (hUCBS or hUCBP), human platelet lysate (HPL) and platelet-rich plasma (PRP).

CN110841109 discloses compositions obtained from human umbilical cord (UC) tissue (UCT) comprising fibrinogen, thrombin, and homogeneous cell population, wherein such UC-tissue is free of blood for improving the cell structure and architecture of the intervertebral disc. This document is silent in what regards to compositions derived from mesenchymal stem cells (MSCs) of human umbilical cord blood (hUCB) plasma comprising regenerative factors.

US2019134099 discloses compositions obtained from human placental tissue for wound healing or tissue regeneration, such as extracellular matrix, growth factors, and cells, including MSCs that are responsible for promoting the healing process in different tissue types. This document is silent in what regards to compositions derived from mesenchymal stem cells (MSCs) of human umbilical cord blood (hUCB) plasma comprising regenerative factors.

Ehrhart, Jared et al. (2018) describes the use of plasma derived from hUCB-plasma for cell-additive or cell-substitute therapeutic for neurodegenerative diseases by promoting mononuclear cells from hUCB (MNC hCBB) viability and reducing apoptotic cell death in vitro, when said cells are cultured with autologous cord blood plasma (CBP). However, this document is also silent in what regards to compositions derived from mesenchymal stem cells (MSCs) of human umbilical cord blood (hUCB) plasma comprising regenerative factors for treating skeletal disorders.

Caseiro AR et al. (2018) discloses a study wherein hUCB-plasma is used as an alternative to animal sera, such as the extensively used foetal bovine serum (FBS) for supplementation of cell culture, namely for MSCs in vitro expansion.

The above documents disclose the preparation of MSCs cultures with supplementation of FBS in vitro.

However, typically hMSCs are not capable of surviving in the absence of FBS or equivalent supplementation, not only because of the important growth factors present, but also the fact that the FBS works as a buffering agent and protects the cells from cytotoxic agents.

The present invention proposes a solution for the above-mentioned problems by disclosing a process for preparation of compositions comprising human umbilical cord blood plasma and a designed specific culture medium to grow these cells in specific conditions.

In this sense, hUCBP proved be an alternative for the FBS supplemented culture medium used in hMSCs in vitro isolation, expansion and cryopreservation and can also as a source of pro-regenerative factors to be used directly without the need of cell transplantation.

### DESCRIPTION OF THE INVENTION

The present invention relates to a process for preparation of compositions comprising human umbilical cord blood plasma and a designed specific culture medium to grow these cells in specific conditions.

The umbilical cord tissue or matrix (Wharton jelly-UCT) and the umbilical cord blood (UCB) are important sources of MSCs and hematopoietic stem cells (HSCs), baring advantages over other sources for being non-invasively collected (previously considered as medical waste).

The umbilical cord blood (UCB) can be safely collected from both human and veterinary species upon birth using it for the development of xeno-free systems for the translation into clinical application in both medical fields including the preparation of hUCBS or hUCBP to be used as culture medium supplementation with pro-regenerative growth factors.

The successful employment of hUCBP as a supplement of cell culture, can result in the preparation of fully autologous cellular therapies, for patients with cryopreserved serum/plasma and cells or tissue.

Alternatively, once the regulatory requisites are established, it can be envisioned for the preparation of allogeneic cellular therapies. This brings increased benefits for a larger number of patients, and turning cellular therapies promptly available, allowing medical action in optimal ‘windows of therapeutic opportunity’ for numerous diseases.

Additionally, it can contribute to the broadening of possible beneficiaries for such therapies, including people that might have not had the chance to preserve autologous samples, and that may be in need of these advanced therapeutic strategies (for example, suffering from age-related of degenerative diseases).

It was identified specific components and factors (proliferative and anti-apoptotic growth factors, immunomodulatory and immunosuppressive cytokines and chemokines) in hUCBP that could have promoting effects on hMSCs in vitro expansion but that can be used instead of the cell transplantation.

The most attractive alternative to FBS is the hUCBP (allogenic or autologous) due to worldwide increase in the number of cryopreserved cord blood units in Public and Private Cord Blood Banks and because nowadays, it is a byproduct that is not used after processing UCB units for cryopreservation purposes.

Moreover, the hMSCs in vitro expansion is essential to achieve appropriate cell numbers for clinical use being in vitro culture scaled-up for clinical application purposes. This has been a challenge nowadays in particular due to high mortality rates e low rates of survive of viable cells when using the traditional supplementation of the culture with FBS.

UCB and UCT samples, have been stored cryopreserved in Private and Public Cord Blood and Tissue banks worldwide in order to obtain hematopoietic stem cells and hMSCs. These cells are used in the treatment of hematopoietic pathologies like some leukemias and in the regeneration of tissues.

However, these cell-based therapies, according to the european Medicines Agency (EMA) and European Commission regulation [EC 1394/2007 of the European Commission], are considered as medicinal products and must be produced in compliance with good manufacturing practices (GMP).

For this purpose, stem cells from either source must be cultured on large scale, and isolated, processed and cryopreserved according to GMP conditions. This implies the usage of xeno-free medium and reagents of clinical grade for the cell culture, for quality assurance and control, and long term rastreability of the samples, where FBS is not a valid and secure alternative.

Currently, there are several concerns on the use of FBS for clinical applications, due to the potential immunogenicity of FBS cultured cells and of xenogeneic proteins present in the cell culture, capable of inducing anaphylactic or arthus-like immune reactions, which have been reported. Complications of using animal sera for in vitro cell culture also include high batch-to-batch variability, unexpected cell growth characteristics, cytotoxicity of uncharacterized factors in the serum, and the risk of possible contamination with virus, prions, bacteria, nanobacteria, mycoplasma, yeast, fungi, and endotoxins. The use of animal sera also implies the sacrifice of a huge number of young animals for blood collection, entailing severe economic, ethical and scientific issues on animal welfare.

These issues reinforce the intensive search for new alternatives to animal sera to better comply with GMP and Food and Drug Administration (FDA) regulations (EMEA CPMP/BWP/1793/02; 2003; EMEA/410/01 rev. 2; 2004).

Therefore, it is long expected a protocol of collection, treatment, expansion and cryopreservation of these cells that is reproducible and viable presenting low risk for the patient, compared with the traditional ones, such as cell transplantation, and the difficulties and complications associated to the engraftment of the allo- or xeno-transplanted cells.

Multiplexing LASER Bead Technology allows the quantification of numerous analyte categories such as cytokines, chemokines and growth factors which can test simultaneously multiple targets in a single assay. When applied to the cultures of the present invention confirmed that the specific culture media (CM) where these cells grow in culture and the hUCBP are very appropriate therapeutic products rich in growth factors comparable to hMSCs local clinical application for tissue regeneration.

The present invention solves the above-mentioned problems by the preparation of a specific culture media (CM) based on MSCs cultured with supplementation of hUCBP instead of FBS, and the application of hUCBP and CM in clinical applications, instead of hMSCs, due to the high concentration of CM and hUCBP in pro-regenerative factors. The CM and the hUCBP can be cryopreserved for latter clinical application.

Due to the above explained, the hUCBP obtained from the hUCB collection, processing and cryopreservation can be directly used in the regeneration of tissues and in the in vitro isolation and hMSCs expansion (and scale-up process for clinical application) as growth factor supplementation.

This also implies that the CM production can be obtained with hUCBP following the xeno-free production of cell-based therapies. The hUCBP or the CM obtained with hUCBP supplementation can be used in regenerative medicine instead of the hMSCs.

Despite current trends demonstrate that in some instances, MSCs remain undifferentiated at the lesion site or in its vicinity, for limited periods of time, or even that only minimal percentages of the MSCs would effectively differentiate and integrate host tissue. Regardless of their differentiation into tissue specific phenotypes, MSCs are often correlated to improved regenerative outcomes. These observations were then attributed to the secretion products of those MSCs. These secretion products are present and highly concentrated in the hUCBP and CM of hMSCs in the present invention.

### DETAILED DESCRIPTION

The present invention relates to a process for preparation of compositions comprising human umbilical cord blood plasma (hUCBP) and a designed specific culture medium grew in specific conditions from hMSCs (CM).

Umbilical cord blood is collected from the umbilical vein by gravity into a simple bag containing citrate-phosphate-dextrose (CPD). Human MSCs from Wharton's jelly umbilical cord (hMSCs) are isolated from the Wharton's jelly of the umbilical cord collected immediately after birth, are isolated using the explant method, prior cryopreserved in liquid nitrogen after a controlled rate cooling protocol. For in vitro culture, the hMSCs are thawed at 37° C. bath and kept in a culture media being maintained in a humidified atmosphere with 5% CO₂.

The UCB is stored at a refrigerated temperatures varying from 4° C. to 6° C. until processing for secretome cryopreservation between −1960° C. and −156° C. that includes a complete separation of concentrated mononuclear cell (MNC) fraction, red blood cells (RBC) fraction and plasma.

The hUCBP can be used as an alternative to FBS in culture media of hMSCs between 6% and 10% of the total volume (V/V) after this process of separation. For that purpose, the cryopreserved hUCBP is thawed at a 37° C. bath and added to the culture media. The hMSCs are maintained in a humidified atmosphere with 5% CO₂ at 37° C. The hMSCs expanded and in vitro cultured using hUCBP as supplement can afterwards produce the CM.

Mesenchymal Stromal/Stem Cells populations in vitro cultured with hUCBP as supplement, depicted characteristic phenotypical markers, presenting ≥92% positive population for CD90, CD105 and CD44, and ≤2% negative marking for CD34, CD11b, CD19, CD45 and MHC II. The differentiation capacity of these MSCs towards three mesodermal lineages was confirmed (osteogenic, condrogenic and adipogenic differention).

Given the paracrine capacity attributed to the hMSCs, their culture medium can be a better suited alternative to MSCs application for tissue regeneration, as it can benefit from the local tissue response to the secreted molecules without the difficulties associated to the engraftment of the transplanted cells. These facts drive the research to the need of CM production.

Isolated hMSCs are expanded and submitted to a Conditioning Protocol. hMSCs are thawed and in vitro expanded being maintained at 37° C. and 95% humidified atmosphere with 5% CO₂ environment. hMSCs are initially maintained in αMEM, with GlutaMAX™, without nucleosides supplemented with 6% to 10% (v/v) hUCBP, 100 IU/ml penicillin, 0.1 mg/ml streptomycin, 2.05 μg/ml amphotericin B and 10 mM HEPES Buffer solution (unconditioned culture medium).

After reaching 80% confluency, regular culture medium (containing hUCBP) is replaced by serum/plasma free DMEM-F12 medium and left for 24 or 48 hours to secrete trophic factor into the plain medium. Ended this time, the CM is collected, and centrifuged. A volume of CM is aliquoted and stored at −80° C. for further analysis. Other is previously concentrated 5 times, reducing the final volume, while expectedly maintaining the total amount of secreted factors.

An extensive panel of soluble factor is quantified by Multiplexing LASER Bead analysis, such as: epidermal growth factor (EGF), eotaxin-1, fibroblast growth factor 2(FGF-2), fms-related tyrosine kinase 3 ligand (Flt-3L), fractalkine, granulocyte colony-stimulating factor (G-CSF), granulocyte macrophage colony-stimulating factor (GM-CSF), GRO (pan), interferon-alpha 2 (IFNα2), interferon-gama (IFNγ), several interleukins (IL-1α, IL-1β, IL-1ra, IL-2, IL-3, IL-4, IL-5, IL-6, IL-7, IL-8, IL-9, IL-10, IL-12 (p40), IL-12 (p70), IL-13, IL-15, IL-17A), interferon gama-induced protein 10 (IP-10), monocyte chemotactic protein-1 (MCP-1), monocyte chemotactic protein-3 (MCP-3), macrophage-derived chemokine (MDC), macrophage inflammatory protein-1 alpha (MIP-1α), macrophage inflammatory protein-1 beta (MIP-1β), platelet-derived growth factor-AA (PDGF-AA), ), platelet-derived growth factor-AB/BB (PDGF-AB/BB), chemokin (C-C motif) ligand 5 (RANTES or CCL5), soluble CD40 ligand (sCD40L), transforming growth factor alpha (TGFα), tumor necrosis factor alpha (TNFα), tumor necrosis factor beta TNFβ, vascular endothelial growth factor A (VEGF-A), and tumor growth factor beta 1, 2 and 3 (TGF-β 1, 2, and 3) [PeGäAmAlMa14, PeIvCaBaMa14, CaIvPeBrMa18, CaPeIvBrMa19].

A pattern of superior secretion by hMSCs cultured using 6% to 10% of hUCBP as supplement is observed throughout, highlighting HGF, IL-8, and MCP-1 expressive quantities when compared with hMSCs in vitro supplemented with FBS.

This evidence shows that CM obtained from in vitro culture and expansion of hMSCs and hematopoietic stem cells (CD34+ cells) or hUCBP, according to the present invention, present better therapeutic options compared to in vivo transplantation of these stem cells. This is because the regenerating tissues can benefit from the local tissue response to the secreted molecules without the difficulties and complications associated to the engraftment of the allo-transplanted or xeno-transplanted cells.

### 1. Preparation of Umbilical Cord Plasma

UCB is collected from the umbilical vein by gravity into a simple bag containing citrate-phosphate dextrose (CPD). This procedure is performed by trained midwives after delivery of the placenta. The UCB is stored at refrigerated temperatures between +4° C. and +6^{a}C until processing for cryopreservation.

1.1. Volume Reduction and Separation of the hUCBP

The system for separation and reduction process of the UCB consists of a microprocessor-controlled device and a disposable closed blood bag set. The device contains different compartments for housing the processing set and flow control optical sensors that are used to achieve the separation of a concentrated mononuclear cell (MNC) fraction of uniform volume. The collected UCB is transferred to the bag set by means of a sterile dock tubing system, through a clot filter and loaded into the device. During the two-step centrifugation, whole blood is separated into three layers that are delivered into a red blood cells (RBC) bag and a freezing bag. Plasma remains in the processing bag which is also the plasma bag. The device fits most standard blood bank centrifuge buckets. The programmed final volume of UCB in the cryopreservation bag is 21 ml. The hUCBP is kept in the plasma bag and also analysed. The UCB and the hUCBP can be cryopreserved in separate bags, after adding 6% (V/V) of Dimethyl-sulfoxide (DMSO) and subject to a controlled cooling program before entering the cryopreservation tank.

### 1.2. Biological Controls of the UCB

The CD34 antigen is present on immature hematopoietic precursor cells and hematopoietic colony-forming cells in bone marrow, peripheral blood and umbilical cord blood, including unipotent and pluripotent progenitor cells. An accurate measure of the CD34⁺ cell count is necessary for dose requirements protocols on stem cell transplantation and stem cells therapies. Total nucleated cells (TNC) count, CD34⁺ cell counts, CD34⁺ cell viability and leucocytes (CD45⁺) viability are determined on samples obtained from the UCB before volume reduction. Microbiological controls are performed after volume reduction and before cryopreservation and tested for microbiological contamination. TNC and the number of white blood cells (WBC) is counted with a haematology autoanalyzer. The CD34⁺ cell number and the CD34⁺ viability are quantified by flow cytometry.

### 2. Preparation of hMSCs Conditioned Media (CM)

hMSCs cryopreserved are in vitro cultured and maintained in a humidified atmosphere with 5% CO₂ at 37° C. The hMSCs thawed at 37° C. bath and expanded exhibit a mesenchymal-like shape with a flat and polygonal morphology when in vitro cultured.

Mesenchymal stem cell medium composed of αMEM, with GlutaMAX™, without nucleosides supplemented with 6% to 10% (v/v) FBS or hUCBP, 100 IU/ml penicillin, 0.1 mg/ml streptomycin, 2.05 μg/ml amphotericin B and 10 mM HEPES Buffer solution is replaced every 48 hours. At 80% confluence, cells are harvested with 0.25% trypsin with EDTA and passed into a new flask for further expansion. The culture medium is supplemented with 6% to 10% of hUCBP, 2 mM glutamine, 100 U/ml of penicillin and 100 μg/ml of streptomycin. Conditioned media (CM) is collected from P4 hMSCs. To obtain the desired CM, 4000 cell/cm² are plated and allowed to grow until reaching a minimum of 80% confluence. At this stage, the commercial medium is removed from the T-flasks and after 5 washing cycles with Dulbecco's Phosphate Buffered Saline 1X (DPBS) without calcium (Ca²⁺) and magnesium (Mg²⁺), Dulbecco's Modified Eagle Medium/Nutrient Mixture (DMEM) supplemented with 100 U/ml of penicillin and 100 μg/ml of streptomycin (Sigma) is added. The cell culture T-flasks are maintained in a humidified atmosphere with 5% CO₂ at 37° C., allowing the adherent cells to be in contact with a serum-free basal medium. The culture media added after the confluence is reached is not renewed, and it is collected at different time points (24 h and 48 h). Upon collection, the CM is frozen at −20° C., being later on thawed to perform Multiplexing LASER Bead analysis. This CM can be cryopreserved in LN₂ to be applied locally after infiltration, in order to promote tissue regeneration. For that purpose, the obtained CM is added of 6% (V/V) of Dimethyl-sulfoxide (DMSO) and subject to a controlled cooling program before entering the cryopreservation tank.

The phenotype of the hMSCs expanded is confirmed by flow cytometry in. order to certify that the hMSCs followed the International Society for Cellular Therapy (ISCT) criteria. Detection is performed with the following antibodies and their respective isotypes: PE anti-human CD105; APC anti-human CD73; PE anti-human CD90; PerCP/Cy5.5 anti-human CD45: FITC anti-human CD34; PerCP/Cy5.5 anti-human CD14; Pacific Blue anti-human CD19 and pacific-blue anti-human HLA-DR. The karyotype of undifferentiated hMSCs is determined in order to certify the absence of neoplastic characteristics in these cells, as well as the chromosomal stability to the cell culture procedures.

### 3. Composition of hUCBP and CM

Multiplexing LASER Bead Technology permits the simultaneous testing of numerous analyte categories such as cytokines, chemokines and growth factors in a single assay and is based on color-coded polystyrene beads. The bead analyzer includes a dual-laser system and a flow-cytometry system. One laser activates the fluorescent dye within the beads which identifies the specific analyte. The second laser excites the fluorescent conjugate (streptavidin-phycoerythrin) that has been bound to the beads during the assay. The amount of the conjugate detected by the analyzer is in direct proportion to the amount of the target analyte. The results are quantified according to a standard curve using 25 μl of hUCBP, unconditioned culture medium and CM. Human Primary Cytokine Array/Chemokine Array 41-Plex Panel is performed to analyze the hUCBP and the CM, including the following cytokines, chemokines and growth factors: epidermal growth factor (EGF), eotaxin-1, fibroblast growth factor 2 (FGF-2), fms-related tyrosine kinase 3 ligand (Flt-3L), fractalkine, granulocyte colony-stimulating factor (G-CSF), granulocyte macrophage colony-stimulating factor (GM-CSF), GRO(pan), interferon-alpha 2 (IFNα2), interferon-gama (IFNγ), several interleukins (IL-1α, IL-1β, IL-1ra, IL-2, IL-3, IL-4, IL-5, IL-6, IL-7, IL-8, IL-9, IL-10, IL-12 (p40), IL-12 (p70), IL-13, IL-15, IL-17A), interferon gama-induced protein 10 (IP-10), monocyte chemotactic protein-1 (MCP-1), monocyte chemotactic protein-3 (MCP-3), macrophage-derived chemokine (MDC), macrophage inflammatory protein-1 alpha (MIP-1α), macrophage inflammatory protein-1 beta (MIP-1β), platelet-derived growth factor-AA (PDGF-AA), ), platelet-derived growth factor-AB/BB (PDGF-AB/BB), chemokin (C-C motif) ligand 5 (RANTES or CCL5), soluble CD40 ligand (sCD40L), transforming growth factor alpha (TGFα), tumor necrosis factor alpha (TNFα), tumor necrosis factor beta TNFβ, vascular endothelial growth factor A (VEGF-A). TGF-β 3-Plex Array Multi-Species is also performed to analyze the hUCBP and the CM, including tumor growth factor beta 1, 2 and 3 (TGF-β 1, 2, and 3). The samples are kept at −20° C. until the analysis was performed.

When compared the composition in growth factors in hUCBP and CM, according to the invention with the typical ones, by Multiplexing LASER Bead Technology it was found that it consists in an adequate and richer alternative therapies with hMSCs since the CM and hUCBP are important sources of growth factors, such as proliferative, chemotactic and immunomodulatory soluble factors (TGF-β, G-CSF, GM-CSF, MCP-1, IL-6, IL-8). As a matter of fact, these growth factors are detected in high concentrations in CM and even higher in hUCBP. On the other hand, the fact that hUCBP is rich in growth factors, can also be used as a substitute of FBS in cell culture medium, for preparation of hMSCs for clinical applications.

Data obtained reinforces the potential use of hUCBP and CM in tissue regeneration and focus the possible use of hUCBP as a substitute for the FBS used in in vitro hMSCs culture. A complete method of preparation of the umbilical cord blood, cryopreservation of the plasma and quantification of the growth factors was developed. Also, the preparation of CM is described as well as its quantification of growth factors. This method improves the biobanking of human samples derived from UCB and UCT, also is simple, easy, does not require expertise in cell culture and allows the use of a by-product of the umbilical cord blood cryopreservation industry.

In the scope of the present invention the term “protocols” is interchangeable with “methods” and defines a combination of techniques and reactants that contribute to the final product result of this invention, such as the collected tissue/samples to be worked with, the designed culture media and the conditions of conservation and preservation of the treated products till the very end, i.e., when are ready to be administered to an individual.

The detailed characterization of hMSCs secretome is becoming particularly relevant because the factors secreted by these cells may be the main effectors of their therapeutic action. This recent paradigm has suggested that the biomolecules synthesized by stem cells may be as important, if not more so, than differentiation of the cells in eliciting functional tissue repair.

This evidence shows that CM obtained from in vitro culture and expansion of hMSCs and hematopoietic stem cells (CD34+ cells) or hUCBP are probably better therapeutic options compared to in vivo transplantation of these stem cells. This is because the regenerating tissues can benefit from the local tissue response to the secreted molecules without the difficulties and complications associated to the engraftment of the allo-transplanted or xeno-transplanted cells.

### List of Abbreviations

CD—Cluster of Differentiation
CM=Conditioned Media
CPD—citrate-phosphate-dextrose
DMEM—Dulbecco's Modified Eagle Medium/Nutrient Mixture
DMSO—Dimethyl-sulfoxide
DPBS—Dulbecco's Phosphate Buffered Saline
EC—European Commission regulation
EMA—European Medicines Agency
FBS—Fetal Bovine Serum
FDA—Food and Drug Administration
hMSCs—human Mesenchymal Stromal/Stem Cells
HPL—human Platelet Lysate
HSCs—Hematopoietic Stem Cells
hUCBP—human Umbilical Cord Blood Plasma
hUCBS—human Umbilical Cord Blood Serum
ISCT—International Society for Cellular Therapy
MNC—mononuclear cell
MSCs—Mesenchymal Stromal/Stem Cells
P4—Passage 4
PRP—Platelet-Rich Plasma
RBC—red blood cells
UC—Umbilical Cord
UCB—Umbilical Cord Blood
UCT—Umbilical Cord Tissue

### EXAMPLES

### Example 1. Collection of the Tissues

Umbilical cord blood (UCB) and native cells are recovered from umbilical cord tissue (UCT) conventionally. Samples have been stored, cryopreserved in Private and Public Cord Blood and Tissue banks in Portugal and worldwide in order to obtain hematopoietic stem cells and hMSCs for cell-based therapies.

### Example 2. Composition of the Culture Media

Culture media enriched in growth factors produced by these hMSCs in expansion (Conditioned medium—CM) is prepared according to the invention.

Isolated hMSCs are expanded and submitted to a Conditioning Protocol to obtain CM. hMSCs are thawed and in vitro expanded being maintained at 37° C. and 95% humidified atmosphere with 5% CO₂ environment. hMSCs are initially maintained in αMEM, with GlutaMAX™, without nucleosides supplemented with 6% to 10% (v/v) hUCBP, 100 IU/ml penicillin, 0.1 mg/ml streptomycin, 2.05 μg/ml amphotericin B and 10 mM HEPES Buffer solution (unconditioned culture medium).

After reaching 80% confluency, regular culture medium (containing hUCBP) is replaced by serum/plasma free DMEM-F12 medium and left for 24 or 48 hours to secrete trophic factor into the plain medium. Ended this time, the CM is collected, and centrifuged. A volume of CM is aliquoted and stored at −80° C. for further analysis. Other is previously concentrated 5 times, reducing the final volume, while expectedly maintaining the total amount of secreted factors.

### Example 3. Pro-Regenerative Factors Composition and Preparation of hUCBP and CM

For this purpose, compositions of the hUCBP and CM from hMSCs obtained from UCT are prepared and characterized important sources of growth factors, and the following results were obtained:

The results gathered from the Human Primary Cytokine Array/Chemokine Array 41-Plex Panel together with the TGF-β 3-Plex Array, allowed us to perform a comprehensive analysis of the cytokines and growth factors included in the hUCBP and CM. In terms of the proliferative and anti-apoptotic factors it is clear that the CM presents an increase in terms of concentration of these factors. This was particularly evident for TGF-β1, EGF, G-CSF, GM-CSF, PDGF-AA and VEGF. Some of these factors like EGF are even absent in unconditioned media. It must be also stated that factors such as TGF-β1 and G-CSF are present in considerable increased concentrations in 48 h conditioning samples. TGF-β1 is associated with stem cell differentiation and protection and notably for its anti-apoptotic effects. G-CSF is known for progenitor cells mobilization and also for its anti-apoptotic effects (revised by Doorn et al. [Doorn J, Moll G, Le Blanc K, van Blitterswijk C, de Boer J (2012) Therapeutic applications of mesenchymal stromal cells: paracrine effects and potential improvements. Tissue Engineering Part B: Reviews 18:101-115].

hUCBP presents very high concentrations of TGFβ (1-3). For example TGF-β1 has a 5-fold lower concentration in the 48 h CM compared to the hUCBP. This is also observed in lower scale, with factors like VEGF, PDGF-AA, PDGF-BB and EGF. The tendency observed for the increased values in CM (vs unconditioned medium) samples is also observed in all tested chemokines and mostly in MCP-1, MCP-3, RANTES, GRO, IL-8. It should be noticed that in hUCBP, RANTES is detected with a concentration higher than 70000 pg/ml, while in CM ranged from 2 to 250 pg/ml. hUCBP also presents higher concentrations of eotaxin and fractakline compared to the CM. Apart from the already referred TGF family, other immunosuppressive/immunomodulatory factors are predominantly high in the CM and hUCBP.

In conclusion, the detailed characterization of hMSCs secretome is becoming particularly relevant because the factors secreted by these cells may be the main effectors of their therapeutic action. The low survival rate of transplanted cells into the damaged tissues has also been proved previously by others. Consequently, the clinical benefits are only transient and attributed mostly to transplanted cell-associated paracrine effects that for example stimulate angiogenesis by stimulating endothelial cell adhesion through chemotactic factors. This recent paradigm has suggested that the biomolecules synthesized by stem cells may be as important, if not more so, than differentiation of the cells in eliciting functional tissue repair. This evidence suggests that CM obtained from the in vitro culture and expansion of hMSCs and hematopoietic stem cells (CD34+ cells) or hUCBP are probably better therapeutic options compared to the in vivo transplantation of these stem cells. This is because the regenerating tissues can benefit from the local tissue response to the secreted molecules without the difficulties and complications associated to the engraftment of the allo-transplanted or xeno-transplanted cells.

1-10. (canceled)

11. A process for preparing a composition characterized by comprising the following steps:
a. providing a sample of human umbilical cord, wherein the Whärton jelly is separated of the umbilical cord matrix,
b. providing a sample of human umbilical cord blood (hUCB) as separated in the previous step a,
c. refrigerating the sample of human umbilical cord blood (hUCBP) of step b at a temperature of 4° C. to 6° C., for a maximum period of 24 h to 96 h,
d. separation and concentration of the mononuclear cell (MNC) fraction, red blood cells (RBC) fraction and human umbilical cord blood plasma (hUCBP) from the total refrigerated human umbilical cord blood (hUCB) of step c by a two-step centrifugation,
e. performing a further separation of the human umbilical cord blood plasma (hUCBP) of step d from the mononuclear cell (MNC) fraction, red blood cells (RBC) fraction, and
f. cryopreservation of the human umbilical cord blood plasma (hUCBP) fraction obtained according to the previous step e at a temperature of −196° C. and −156° C. after addition of 6% (V/V) of Dimethyl-sulfoxide (DMSO).

12. A process for preparing a composition according to claim 11, wherein:
a. step a further comprises a step a1 on which a population of mesenchymal stem cells (MSCs) is isolated from the Wharton jelly of the umbilical cord matrix, and
b. said isolated mesenchymal stem cells (MSCs) of step al are grown by plating an average amount of 4000 MSCs/cm² in a base culture medium (CMb) at a temperature of 25° C. to 45° C., preferably at a temperature of 30° C. to 40° C., more preferably at a temperature of 35° C. to 37° C. in bath and being maintained in a humidified atmosphere with 5% CO₂, until a minimum of 80% confluence is achieved, and
the base culture medium (CMb) comprises:
αMEM, with GlutaMAX™, without nucleosides, with addition of 100 IU/ml penicillin, 0.1 mg/ml streptomycin, 2.05 μg/ml amphotericin B and 10 mM HEPES Buffer solution, with
human umbilical cord blood plasma (hUCBP) previously separated from the total umbilical cord blood, wherein said hUCBP is present in the final base culture medium (CMb) in an amount of 6% and 10% of the total volume (V/V), and
said base culture medium (CMb) is free of foetal bovine serum (FBS),
c. Replacement of the base culture medium (CMb) of the culture of the previous step b by a conditioning culture medium (CCM), wherein the used CMb is removed from said culture and the CCM is added in an amount of 25% (V/V) in relation to the initial amount of CMb, and
the conditioning culture medium (CCM) comprises:
Dulbecco's Modified Eagle Medium with Ham's F-12 (DMEM-F12),
100 IU/ml penicillin, 0.1 mg/ml streptomycin, 2.05 μg/ml amphotericin B, and
10 mM (4-(2-hydroxyethyl)-1-piperazineethanesulfonic acid) (HEPES) buffer solution,
d. Culturing the resulting MSCs population of step c in the conditioning culture medium (CCM) for 24 h to 48 hours.

13. A process for preparing a composition according to claim 12, wherein the culture of population of mesenchymal stem cells (MSCs) including the respective conditioning culture medium (CCM) is subject to the addition of an amount of 6% (V/V) of Dimethyl-sulfoxide (DMSO) after achieving a minimum of 80% confluence.

14. A process for preparing a composition according to claim 13, wherein the culture of population of mesenchymal stem cells (MSCs) including the respective conditioning culture medium (CCM) is concentrated by centrifugation and then refrigerated at a temperature of 4° C. to 6° C., for a period of 24 h to 96 h.

15. A process for preparing a composition according to claim 14, wherein the culture of population of mesenchymal stem cells (MSCs) is concentrated 5 times before being refrigerated at a temperature of 4° C. to 6° C., for a period of 24 h to 96 h.

16. A process for preparing a composition according to claim 15, wherein the culture of population of mesenchymal stem cells (MSCs) is further cryopreserved at a temperature of or below −80° C., preferably at a temperature of or below −140° C..

17. A composition prepared by the process of claim 11, characterized by comprising the following molecules:
epidermal growth factor (EGF), eotaxin-1, fibroblast growth factor 2 (FGF-2), fms-related tyrosine kinase 3 ligand (Flt-3L), fractalkine, granulocyte colony-stimulating factor (G-CSF), granulocyte macrophage colony-stimulating factor (GM-CSF), GRO(pan), interferon-alpha 2 (IFNα2), interferon-gama (IFNγ), several interleukins (IL-1α, IL-1β, IL-1ra, IL-2, IL-3, IL-4, IL-5, IL-6, IL-7, IL-8, IL-9, IL-10, IL-12 (p40), IL-12 (p70), IL-13, IL-15, IL-17A), interferon gama-induced protein 10 (IP-10), monocyte chemotactic protein-1 (MCP-1), monocyte chemotactic protein-3 (MCP-3), macrophage-derived chemokine (MDC), macrophage inflammatory protein-1 alpha (MIP-1α), macrophage inflammatory protein-1 beta (MIP-1), platelet-derived growth factor-AA (PDGF-AA),), platelet-derived growth factor-AB/BB (PDGF-AB/BB), chemokine (C-C motif) ligand 5 (RANTES or CCL5), soluble CD40 ligand (sCD40L), transforming growth factor alpha (TGFα), tumor necrosis factor alpha (TNFα), tumor necrosis factor beta TNFβ, vascular endothelial growth factor A (VEGF-A) to be applied in medicine in cell-based therapies.

18. A composition according to claim 17, wherein the cell-based therapy is related to tissue regeneration processes or to inflammatory processes associated to tissue regeneration processes.

19. A composition according to claim 17, wherein cell-based therapy is related to skeletal disorders, such as joint disorders, disorders of connective tissue, bone diseases and osteoporosis

20. A composition according to claim 17, wherein the medicine cell-based therapy is related to transplantation therapies.
